(19) United States
(12) Patent Application Publication
(10) Pub. No.: US 2016/0168053 A1
(43) Pub. Date: Jun. 16, 2016

(54) INTEGRATED PROCESS FOR NORMAL PARAFFIN ALKYLATION

(71) Applicant: UOP LLC, Des Plaines, IL (US)

(72) Inventors: Tom N. Kalnes, LaGrange, IL (US); Stuart Smith, Lake Zurich, IL (US); Alakananda Bhattacharyya, Glen Ellyn, IL (US); Mary Wier, Schaumburg, IL (US)

(21) Appl. No.: 14/567,749

(22) Filed: Dec. 11, 2014

(51) Int. Cl. C07C 6/10; C07C 2/60; C07C 2/62

(52) U.S. Cl. CPC C07C 6/10; C07C 2/62; C07C 2/60; C07C 2527/173; C07C 2527/1206; C07C 2527/08; C07C 2527/054; C07C 2527/13

(57) ABSTRACT

An integrated alkylation and disproportionation process and apparatus are described. n-C₄ and n-C₅ are routed to a disproportionation reaction zone for conversion to iso-C₄ and C₆₊ isoparaffin-rich product. The iso-C₄ is routed to an alkylation reaction zone and reacted with refinery propylene and butenes to produce alkylate product. The C₆₊ isoparaffin-rich product and alkylate product are recovered. Unconverted iso-C₄ and/or olefins are recycled to the alkylation reaction zone, and unconverted n-C₄ and n-C₅ are recycled to the disproportionation reaction zone.

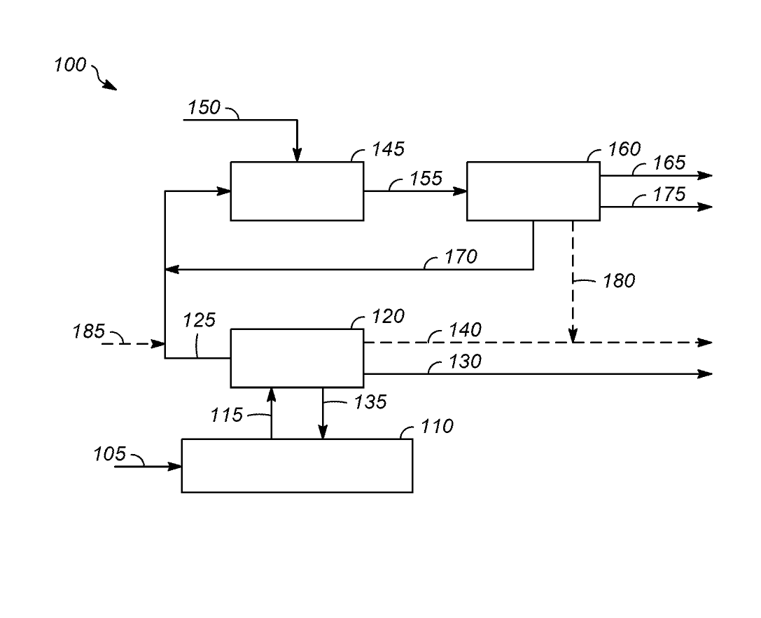

### BACKGROUND OF THE INVENTION

The use of catalytic alkylation processes to produce branched hydrocarbons having properties that are suitable for use as gasoline blending components is well known in the art. Generally, the alkylation of olefins, such as butenes, by isoparaffins, such as isobutane, is accomplished by contacting the reactants with an acid catalyst to form a reaction mixture, settling said mixture to separate the catalyst from the hydrocarbons, and further separating the hydrocarbons, for example, by fractionation, to recover the alkylation reaction product. Normally, the alkylation reaction product is referred to as “alkylate”, and preferably contains hydrocarbons having to 7-9 carbon atoms. In order to have the highest quality gasoline blending stock, it is preferred that hydrocarbons formed in the alkylation process be highly branched.

Due to the increased use of shale crude and tar sands, refiners must now accommodate a growing amount of normal paraffins, such as n-butanes and n-pentanes in the feedstock. Finally, some refineries are trying to manage an increasing amount of light olefin byproducts, such as propylene, produced in existing fluid catalytic cracking (FCC) units.

There is a need for a more flexible process that can accept these feeds without requiring additional isobutane from an external source.

### SUMMARY OF THE INVENTION

One aspect of the invention is a process for normal paraffin alkylation. In one embodiment, the process includes introducing a feed comprising n-C₄ and n-C₅ paraffins to a disproportionation reaction zone in the presence of a disproportionation catalyst under disproportionation conditions to form a disproportionation mixture comprising iso-C₄ and C₆₊ disproportionation products and unreacted n-C₄ and n-C₅ paraffins. The disproportionation mixture is separated in the disproportionation separation zone into at least an iso-C₄-rich stream, a C₆₊ isoparaffin-rich stream, and a stream rich in unreacted n-C₄ and n-C₅ paraffins. The iso-C₄-rich stream and an olefin feed stream comprising at least one of ethylene, propylene, and butenes are introduced into an alkylation reaction zone in the presence of an alkylation catalyst under alkylation conditions to produce an alkylation mixture comprising alkylate and unreacted iso-C₄ paraffins. The alkylation mixture is separated in an alkylation separation zone into at least an alkylate-rich stream, and a stream rich in unreacted iso-C₄ paraffins. The stream rich in unreacted n-C₄ and n-C₅ paraffins from the disproportionation separation zone is recycled to the disproportionation reaction zone. The stream rich in unreacted iso-C₄ paraffins from the alkylation separation zone is recycled to the alkylation reaction zone, and at least one of the C₆₊ isoparaffin-rich stream from the disproportionation separation zone and the alkylate-rich stream from the alkylation separation zone is recovered.

Another aspect of the invention is an apparatus for normal paraffin alkylation. In one embodiment, the apparatus includes a disproportionation reaction zone having an inlet and an outlet; a disproportionation separation zone having an inlet and at least one outlet, the inlet of the disproportionation separation zone being in fluid communication with the outlet of the disproportionation reaction zone, an outlet of the disproportionation separation zone being in fluid communication with the inlet of the disproportionation reaction zone; an alkylation reaction zone having at least one inlet and at least one outlet, an inlet of the alkylation reaction zone being in fluid communication with an outlet of the disproportionation separation zone; and an alkylation separation zone having an inlet and at least one outlet, the inlet of the alkylation separation zone being in fluid communication with the outlet of the alkylation reaction zone, an outlet of the alkylation separation zone being in fluid communication with the inlet of the alkylation reaction zone.

### BRIEF DESCRIPTION OF THE DRAWING

The FIGURE is an illustration of one embodiment of a process according to the present invention.

### DETAILED DESCRIPTION OF THE INVENTION

By integrating a process for converting n-C₄ and n-C₅ to iC₄- and C₆₊ products with an acid catalyzed alkylation process, it is possible to increase conversion of these lower value feedstocks to higher value gasoline blendstocks.

In the integrated process, n-C₄ and n-C₅ are routed to a catalytic disproportionation reaction zone where they are converted to iso-C₄ and C₆₊ isoparaffin-rich co-product. The reaction products are then separated, and the iso-C₄ is routed directly (i.e., without intermediate storage) to an integrated acid catalyzed alkylation reaction zone. In the simplest form of the process, the iso-C₄ is routed as a vapor from the disproportionation separation zone to the alkylation reaction zone.

In the disproportionation product separation zone, unconverted n-C₄ and n-C₅ are recovered as liquids and recycled back to the disproportionation reaction zone to increase conversion.

In the alkylation reaction zone, the iso-C₄ derived from the normal paraffins is reacted with refinery propylene and butenes to produce alkylate product. The co-products of this reaction may include additional light naphtha (C₅ and C₆), C₉₊ compounds and lighter paraffins. The alkylate products are separated in an alkylation product separation zone. Unconverted iso-C₄ and/or olefins are recycled to the alkylation reaction zone.

The integrated process allows conversion of excess n-C₄ and n-C₅ in the summer months to lower RVP gasoline. It can allow the refinery to avoid purchasing iso-C₄ when the alkylation capacity is limited by iso-C₄ availability. In addition, it can expand the alkylation process by allowing the processing of propylene-rich feed from fluid catalytic cracking (FCC) reaction zones because of the presence of the disproportionation reaction zone.

The process also involves the in situ production of additional iso-C₄, which will allow greater utilization of refinery propylene in the production of high octane, low Reid Vapor Pressure (RVP) alkylate.

As used herein, the term “stream” can be a stream including various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C₁, C₂, C₃ . . . Cₙ where “n” represents the number of carbon atoms in the one or more hydrocarbon molecules. Additionally, characterizing a stream as, e.g., an “olefin stream” can mean a stream including or rich in at least one olefin.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally about 30%, preferably about 50%, and optimally about 70%, by mole, of a compound or class of compounds in a feed, an effluent, or a stream.

As used herein, the term “substantially” can mean an amount of at least generally about 80%, preferably about 90%, and optimally about 99%, by mole, of a compound or class of compounds in a feed, an effluent, or a stream.

As used herein, the term “vapor” can mean a gas or a dispersion that may include or consist of one or more hydrocarbons.

As used herein, the term “vaporization” can mean using at least one of heat and pressure to change at least a portion of a liquid to a gas optionally forming a dispersion, such as a gas entraining at least one of liquid and solid particles.

As used herein, the term “communicating” may mean two objects capable of receiving, directly or indirectly, a substance transmitted from one to the other.

As used herein, the term “hydrogen fluoride” can include at least one of a hydrogen fluoride or a hydrofluoric acid. Generally, a hydrofluoric acid is a solution of a hydrogen fluoride in water, where the hydrogen fluoride can disassociate and may form ions of H₃O⁺, H⁺, FHF⁻, and F⁻. The term includes anhydrous hydrogen fluoride.

As used herein, the term “about” means within 10% of the value, or within 5%, or within 1%.

As depicted, process flow lines in the FIGURES can be referred to as lines, pipes, spargers, feeds, effluents, or streams. Particularly, a line, a sparger, or a pipe can contain one or more feeds, effluents, or streams, and one or more feeds, effluents, and streams can be contained by a line, a sparger, or a pipe. Generally, a sparger is a pipe forming a plurality of holes to improve dispersing of material from inside the pipe.

As illustrated in the process 100 shown in the FIGURE, a paraffin feed 105 comprising n-C₄ and n-C₅ is introduced into disproportionation reaction zone 110.

The disproportionation of paraffins involves reacting two moles of hydrocarbon to form one mole each of two different products, one having a carbon count greater than the starting material and the other having a carbon count less than the starting material. The total number of moles in the system remains the same throughout the process, but the products have different carbon counts from the reactants. Additional secondary disproportionation-type reactions can occur in which two hydrocarbons having different carbon numbers react to form two different hydrocarbons having different carbon numbers from those of the feed (e.g., pentane and octane reacting to form hexane and heptanes or pentane and hexane reacting to form butane and heptane). For a feed of Cₓ, the disproportionation products include Cₓ⁺ hydrocarbons and Cₓ⁻ hydrocarbons.

The feed comprising n-C₄ and n-C₅ can be obtained from natural gas liquids (NGLs), liquefied petroleum gas (LPGs), light straight-run naphtha, light naphtha, light natural gasoline, light reformate, light raffinate from aromatics extraction, light cracked naphtha, butanes, normal-butane concentrate, field butanes and the like.

The disproportionation catalyst can be any suitable liquid or solid disproportionation catalyst, including, but not limited to, hydrofluoric acid (HF), sulfuric acid (H₂SO₄), fluorosulfonic acids, acidic ionic liquids, halides of Group III metals, zeolites, alumina, aluminosilicates, and solid acid catalysts.

Typical disproportionation operating conditions include a temperature in the range of about 10° C. to about 300° C., or about 50° C. to about 300° C., a pressure in the range of about 0.1 MPa (g) to about 13.8 MPa (g), and a liquid hourly space velocity (LHSV) is generally in the range of about 0.1 hr⁻¹ to about 1000 hr⁻¹, depending on the catalyst used. Disproportionation processes are described in U.S. Pat. Nos. 6,573,416, and 6,423,880, for example.

By acidic ionic liquid, we mean an ionic liquid capable of catalyzing reactions typically carried out with an acid. As known in the art, acids such as sulfuric acid and hydrofluoric acid are often used to catalyze these reactions. These reactions include, e.g. alkylation, oligomerization, isomerization, disproportionation, and reverse disproportionation. Oftentimes the acids employed in these reactions have Hammett acidity functions (H₀) less than 7, or less than 5, or less than 3, or less than 0, or less than −3, or less than −5, or less than −7, or less than −9. If the ionic liquid does not possess an acidic proton in its structure (e.g. 1-butyl-3-methylimidazolium heptachloroaluminate), addition of an exogenous acid is acceptable, provided the Hammett acidity function (H₀) of the added acid is less than 7 within the ionic liquid, or less than 5, or less than 3, or less than 0, or less than −3, or less than −5, or less than −7, or less than −9. Acidic chloroaluminate-containing ionic liquids have a molar ratio of Al to cation greater than 1.

The ionic liquid can be any acidic ionic liquid. There can be one or more ionic liquids. The ionic liquid comprises an organic cation and an anion. Suitable cations include, but are not limited to, nitrogen-containing cations and phosphorus-containing cations. Suitable organic cations include, but are not limited to:

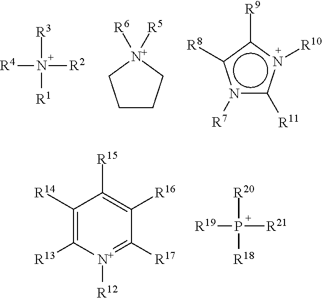

where R¹-R²¹ are independently selected from C₁-C₂₀ hydrocarbons, C₁-C₂₀ hydrocarbon derivatives, halogens, and H. Suitable hydrocarbons and hydrocarbon derivatives include saturated and unsaturated hydrocarbons, halogen substituted and partially substituted hydrocarbons and mixtures thereof. C₁-C₈ hydrocarbons are particularly suitable. Lactamium based ionic liquids can also be used, such as those described in U.S. Pat. No. 8,709,236, U.S. application Ser. No. 14/271,308, entitled Synthesis of Lactam Based Ionic Liquids, filed May 6, 2014, and U.S. application Ser. No. 14/271,319, entitled Synthesis of N-Alkyl Lactam Based Ionic Liquids, filed May 6, 2014, each of which is incorporated herein by reference.

The anion can be derived from halides, typically halometallates, and combinations thereof. The anion is typically derived from metal and nonmetal halides, such as metal and nonmetal chlorides, bromides, iodides, fluorides, or combinations thereof. Combinations of halides include, but are not limited to, mixtures of two or more metal or nonmetal halides (e.g., AlCl₄⁻ and BF₄⁻), and mixtures of two or more halides with a single metal or nonmetal (e.g., AlCl₃Br⁻). In some embodiments, the metal is aluminum, with the mole fraction of aluminum ranging from 0<Al<0.25 in the anion. Suitable anions include, but are not limited to, AlCl₄⁻, Al₂Cl₇⁻, Al₃Cl₁₀⁻, AlCl₃Br⁻, Al₂Cl₆Br⁻, Al₃Cl₉Br⁻, AlBr₄⁻, Al₂Br₇⁻, Al₃Br₁₀⁻, GaCl₄⁻, Ga₂Cl₇⁻, Ga₃Cl₁₀⁻, GaCl₃Br⁻, Ga₂Cl₆Br⁻, Ga₃Cl₉Br⁻, CuCl₂⁻, Cu₂Cl₃⁻, Cu₃Cl₄⁻, ZnCl₃⁻, FeCl₃⁻, FeCl₄⁻, Fe₃Cl₇⁻, PF₆⁻, and BF₄⁻.

One suitable process and catalyst are described in application Ser. No. 14/562,390, entitled DISPROPORTIONATION OF HYDROCARBONS USING SOLID ACID CATALYSTS, (Attorney Docket No. H0043220-8310), filed Dec. 5, 2014, which is incorporated herein by reference. The catalyst comprises a refractory inorganic oxide having a metal halide dispersed thereon. There can optionally be a Group VIII metal component dispersed thereon. The reaction takes place in the presence of hydrogen and a chloride promoter. Suitable disproportionation reaction conditions for this catalyst include a temperature in the range of about 100° C. to about 300° C. The pressure is generally in the range of about 0 MPa (g) to about 13.8 MPa (g). The liquid hourly space velocity (LHSV) is generally in the range of about 0.25 hr⁻¹ to about 10 hr⁻¹.

The disproportionation reaction produces a disproportionation reaction mixture typically including C₃₋, iso-C₄ and C₆₊ disproportionation products, and unreacted n-C₄ and n-C₅ paraffins. There will also be some isomerization of the n-C₄ and n-C₅ to iso-C₄ and iso-C₅.

The disproportionation effluent 115 containing the disproportionation products, unreacted feed, and any isomerization products is introduced into the disproportionation separation zone 120. The disproportionation separation zone 120 can be any suitable separation zone, such as a fractionation column. If an ionic liquid catalyst is used, the disproportionation separation zone 120 may include a gravity settler upstream of the fractionation column to remove the ionic liquid.

The disproportionation effluent 115 can be separated into at least two streams. Typically, the disproportionation effluent 115 will be separated into at least an iso-C₄-rich stream 125, a C₆₊ isoparaffin-rich stream 130, and a stream rich in unreacted n-C₄ and n-C₅ paraffins 135, which will also contain iso-C₅ from the isomerization that occurs. Additional streams could also be formed, including a C₃₋ stream 140.

The C₆₊ isoparaffin-rich stream 130 can be recovered and used for gasoline blending, if desired. Alternatively, it could be used as feed for a reformer.

The stream rich in unreacted n-C₄ and n-C₅ paraffins (and containing the iso-C₅) 135 is recycled to disproportionation reaction zone 110 to improve conversion.

The C₃₋ stream 140 can be used as feed for a steam cracker, steam reformer, dehydrogenation reactor, oxidative dehydrogenation reactor, or used as fuel gas.

The iso-C₄-rich stream 125 is sent to the alkylation reaction zone 145 without intermediate storage. In some processes, the iso-C₄-rich stream 125 is a vapor stream.

An olefin feed stream 150 comprising at least one of ethylene, propylene, and butenes is also introduced into the alkylation reaction zone 145. The olefin feed stream 150 can be obtained from an FCC unit, steam cracker, or dehydrogenation reactor, for example.

The olefins are alkylated by the iso-C₄ for production of high octane alkylate hydrocarbons boiling in the gasoline range and which are suitable for use in gasoline motor fuel. The alkylate hydrocarbon product comprises a major portion of highly branched high-octane aliphatic hydrocarbons having at least seven carbon atoms and less than 10 carbon atoms.

In order to improve selectivity of the alkylation reaction toward the production of the desirable highly branched aliphatic hydrocarbons having seven or more carbon atoms, a substantial stoichiometric excess of isoparaffin hydrocarbons is desirable in the reaction zone. In the alkylation process of the present invention, employing isoparaffins to olefin molar ratios in typically in excess of about 1:1, usually about 4:1 to about 100:1, or about 4:1 to about 70:1, or about 2:1 to about 25:1, or about 5:1 to about 20:1. Generally, the greater the isoparaffins to olefin ratio in an alkylation reaction, the better the results in alkylate quality.

Typically, the alkylation catalyst can include hydrogen fluoride, a sulfuric acid, a hydrofluoric acid, fluorosulfonic acids, a phosphoric acid, a metal halide (typically in conjunction with a Brönsted acid co-catalyst), or other suitable alkylation catalyst.

Alkylation reaction temperatures are typically in the range of from about 5° C. to about 150° C. Lower temperatures favor alkylation reaction of isoparaffins with olefins over competing olefin side reactions such as oligomerization and polymerization. However, overall reaction rates decrease with decreasing temperatures. Temperatures within the given range, and preferably in the range of from about 30° C. to about 130° C., provide good selectivity for alkylation of isoparaffins with olefins at commercially attractive reaction rates.

Reaction pressures in the alkylation reaction zone may range from pressures sufficient to maintain reactants in the liquid phase to about 1.5 MPa (g). Reactant hydrocarbons may be normally gaseous at alkylation reaction temperatures. Reaction pressures in the range of from about 276 kPa (g) (40 psig) to about 1.1 MPa (g) (160 psig) are suitable. With all reactants in the liquid phase, increased pressure has no significant effect upon the alkylation reaction.

When ionic liquid catalysts are used, the temperature is typically in the range of about −20° C. to the decomposition temperature of the ionic liquid, or about −20° C. to about 100° C., for example. The pressure is typically in the range of atmospheric (0.1 MPa (g)) to about 8.0 MPa (g), or about 0.3 MPa (g) to about 2.5 MPa (g).

Contact times for hydrocarbon reactants in an alkylation reaction zone, in the presence of the alkylation catalyst composition of the present invention generally should be sufficient to provide for essentially complete conversion of olefin reactants in the alkylation zone. Preferably, the contact time is in the range of from about 0.05 minute to about 60 minutes.

The heat generated by the reaction can be eliminated using any of the means known to the skilled person.

The alkylation reaction produces an alkylation reaction mixture typically containing alkylate, unreacted iso-C₄ paraffins, C₅ and C₆ products, and C₃₋ products.

The alkylation effluent 155 containing the alkylate products, unreacted feed, and other products is introduced into the alkylation separation zone 160. The alkylation separation zone 160 can be any suitable separation zone, such as a fractionation column. If an ionic liquid catalyst is used, the alkylation separation zone 160 may include a gravity settler upstream of the fractionation column to remove the ionic liquid.

The alkylation effluent 155 can be separated into at least two streams. Typically, the alkylation effluent 155 will be separated into at least an alkylate-rich stream 165, a stream rich in unreacted iso-C₄ 170. Additional streams could also be formed, including a stream rich in C₅ and C₆ products 175, and a C₃₋ stream 180.

The alkylate-rich stream 165 can be blended with gasoline.

The stream rich in unreacted iso-C₄ 170 can be recycled to the alkylation reaction zone to increase the conversion.

The stream rich in C₅ and C₆ products 175 can be blended into gasoline.

The C₃₋ stream 180 can be used as feed for a steam cracker, for a dehydrogenation reactor or used as fuel gas. It can be combined with the C₃₋ stream 140 from the disproportionation separation zone 120, if desired.

In some embodiments, a stream of make-up iso-C₄ 185 can be added to the alkylation reaction zone 145, either directly or by mixing with the iso-C₄-rich stream 125 from the disproportionation reaction zone 110 or the recycle stream rich in unreacted iso-C₄ 170.

The integrated process may be carried out either as a batch, semi-batch, or continuous type of operation, although, it is preferred for economic reasons to carry out the process continuously. It has been generally established that in disproportionation and alkylation processes, the more intimate the contact between the feedstock and catalyst, the better the quality of disproportionation and alkylate product obtained. With this in mind, the present process, when operated as a batch operation with a liquid catalyst, is characterized by the use of vigorous mechanical stirring or shaking of the reactants and catalysts.

In continuous operations, in one embodiment, reactants may be maintained at sufficient pressures and temperatures to maintain them substantially in the liquid phase and then continuously forced through dispersion devices into the disproportionation and/or alkylation reaction zones. The dispersion devices can be jets, nozzles, porous thimbles and the like. The reactants are subsequently mixed with the catalyst by conventional mixing means such as mechanical agitators or turbulence or other general means in the flow system. After a sufficient time, the product can then be continuously separated from the catalyst and withdrawn from the reaction system while the partially spent catalyst is recycled to the reactor. If desired, a portion of the catalyst can be continuously regenerated or reactivated by any suitable treatment and returned to the alkylation reactor.

### EXAMPLES

### Example 1

### Catalyst

The catalyst was a chlorided alumina catalyst containing platinum made for example by U.S. Pat. No. 5,004,859. The concentration of platinum was in the range of 0.002 wt. % to 2 wt. %, the chloride concentration is in the range of 0.1 to 10 wt. % and the alumina phase was one of alpha, gamma, eta or theta.

### Example 2

### Experimental Set Up

The catalytic reactions were typically run using a ⅞″ inner diameter stainless steel tube reactor. Prior to catalyst loading, the reactor was dried by heating the reactor to at least 150° C. with a three-zone clam shell furnace under a stream of flowing nitrogen for at least four hours. After the drying procedure was completed, the reactor was cooled to ambient temperature, connected to a nitrogen line, and the reactor opened under flowing nitrogen. The reactor was inserted through a hole in a nitrogen glovebag, and the connection of the glovebag with the reactor was sealed with electrical tape. The top of the open reactor was enclosed within a glovebag and had nitrogen blowing through it. The catalyst from Example 1 was loaded under nitrogen in the glovebag to the reactor under this positive flow of nitrogen. The reactor was sand packed with 50-70 mesh sand, the sand having been previously calcined to 700° C. for 7 h. Typically, 40 ccs of catalyst was loaded into the reactor, and the reaction was run downflow. The feed had a 1.4 MPa(g) (210 psig) hydrogen header and the concentration of dissolved hydrogen in the feed was determined from the literature values reported in the IUPAC Solubility Data Series volumes 5/6 “Hydrogen and Deuterium” (1981) for pentane and butane. It was assumed that the value for pentane would remain constant for the iC5, iC5/nC5 and iC5/nC5/cyclopentane (CP) feeds. The feed was passed through a high surface sodium dryer prior to introduction to the reactor and was added to the reactor using a pump. A second pump controlled the chloride addition rate. The chloride was dissolved in the feed, and the chloride source (2-chlorobutane) had previously been dried with activated 3A molecular sieves. The two feed streams were introduced to the reactor by joining the two separate feed streams with a Tee connector immediately prior to their introduction to the reactor. The temperature was measured using K-type thermocouples, and the pressure was controlled by means of a backpressure regulator. The effluent was sent directly to an Agilent 6890N gas chromatograph (GC), and the product was analyzed by means of flame ionization detection. A 60 m, 0.32 mm inner diameter, 1.0 m film thickness DB-1 column was used. The initial oven temperature was 40° C., with a 4 minute hold time at this temperature. The oven was then ramped to 135° C. at a 5° C./min ramp rate, and the program was completed once this temperature reached. The GC inlet was 250° C. with a hydrogen carrier gas. The product was then sent directly to a product charger and collected.

### Example 3

### Disproportionation of iC5

The catalytic reaction was run according to the procedure outlined above. The conditions and results are listed in Table 1 below and demonstrate that the presence of small amounts of hydrogen increase the stability of the catalyst.

TABLE 1
Disproportionation of iC5

| TOS (h) | 15 | 20 | 25 |
|---|---|---|---|
| T (° C.) | 172 | 172 | 172 |
| P (psig) | 608 | 610 | 611 |
| Cl (ppm) | 1600 | 1600 | 1600 |
| LHSV (hr⁻¹) | 1.0 | 1.0 | 1.0 |
| H₂/HC ^{a} | 0.02 | 0.02 | 0.02 |
| H₂/Cl ^{b} | 5 | 5 | 5 |
| % iC5 Conv.^{c} | 52 | 53 | 54 |
| % C5P Conv.^{d} | 41 | 42 | 42 |
| % Selec. Disp. ^{e} | 80 | 80 | 77 |
| Compound | | | |
| Methane | 0.00 | 0.00 | 0.00 |
| Ethane | 0.00 | 0.00 | 0.00 |
| Propane | 0.53 | 0.62 | 0.63 |
| iC4 | 21.07 | 21.67 | 20.18 |
| nC4 | 2.54 | 2.54 | 2.51 |
| iC5 | 48.28 | 47.06 | 45.62 |
| nC5 | 10.51 | 10.89 | 12.36 |
| 22DMB | 1.06 | 1.08 | 1.34 |
| 23DMB | 1.77 | 1.75 | 1.79 |
| 2MP | 5.81 | 5.73 | 5.91 |
| 3MP | 3.45 | 3.41 | 3.55 |
| nC6 | 1.49 | 1.47 | 1.63 |
| C7P | 2.32 | 2.42 | 2.45 |
| C8+ | 1.17 | 1.37 | 1.40 |
| Unknown | 0.00 | 0.00 | 0.00 |

^{a} Molar ratio of hydrogen to hydrocarbon in feed,
^{b} molar ratio of hydrogen to chloride,
^{c}% iC5 Conv. = 100 − wt. % iC5,
^{d}% C5P Conv. = 100 − wt. % iC5 − wt. % nC5 and
^{e} % Selec. Disp. = (wt. % C₄₋ + wt. % C₆₊)/(100 − wt. % iC5) × l00.

### Example 4

### Disproportionation of nC5

The catalytic reaction was run according to the procedure outlined above. The conditions and results are listed in Table 2 below and demonstrate that the disproportionation of nC5 readily occurs with these types of catalysts and that with small amounts of hydrogen being present, the catalyst stability is increased.

TABLE 2
Disproportionation of nC5

| TOS (h) | 8 | 13 | 28 |
|---|---|---|---|
| T (° C.) | 176 | 175 | 171 |
| P (psig) | 619 | 618 | 622 |
| Cl (ppm) | 1600 | 1600 | 1600 |
| LHSV (hr⁻¹) | 1.0 | 1.0 | 1.0 |
| H₂/HC ^{a} | 0.02 | 0.02 | 0.02 |
| H₂/Cl ^{b} | 5 | 5 | 5 |
| % nC5 Conv.^{c} | 69 | 68 | 67 |
| % C5P Conv.^{d} | 35 | 35 | 33 |
| % Selec. Disp. ^{e} | 50 | 52 | 50 |
| Compound | | | |
| Methane | 0.00 | 0.00 | 0.00 |
| Ethane | 0.00 | 0.00 | 0.00 |
| Propane | 0.81 | 0.81 | 0.66 |
| iC4 | 15.49 | 15.83 | 15.19 |
| nC4 | 3.70 | 3.32 | 2.54 |
| iC5 | 34.05 | 32.70 | 33.60 |
| nC5 | 31.26 | 32.06 | 33.18 |
| 22DMB | 1.47 | 1.35 | 1.33 |
| 23DMB | 1.30 | 1.36 | 1.37 |
| 2MP | 4.09 | 4.25 | 4.25 |
| 3MP | 2.54 | 2.64 | 2.62 |
| nC6 | 1.44 | 1.45 | 1.32 |
| C7P | 2.46 | 2.64 | 2.44 |
| C8+ | 1.39 | 1.60 | 1.50 |
| Unknown | 0.00 | 0.00 | 0.00 |

^{a} Molar ratio of hydrogen to hydrocarbon in feed,
^{b} molar ratio of hydrogen to chloride,
^{c}% nC5 Conv. = 100 − wt. % nC5,
^{d}% C5P Conv. = 100 − wt. % iC5 − wt. % nC5 and
^{e} % Selec. Disp. = (wt. % C₄₋ + wt. % C₆₊)/(100 − wt. % nC5) × 100.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for normal paraffin alkylation comprising:
introducing a feed comprising n-C₄ and n-C₅ paraffins to a disproportionation reaction zone in the presence of a disproportionation catalyst under disproportionation reaction conditions to form a disproportionation mixture comprising iso-C₄ and C₆₊ disproportionation products and unreacted n-C₄ and n-C₅ paraffins;
separating the disproportionation mixture in a disproportionation separation zone into at least an iso-C₄-rich stream, a C₆₊ isoparaffin-rich stream, and a stream rich in unreacted n-C₄ and n-C₅ paraffins;
introducing the iso-C₄-rich stream and an olefin feed stream comprising at least one of ethylene, propylene, and butenes into an alkylation reaction zone in the presence of an alkylation catalyst under alkylation reaction conditions to produce an alkylation mixture comprising alkylate and unreacted iso-C₄ paraffins;
separating the alkylation mixture in an alkylation separation zone into at least an alkylate-rich stream, and a stream rich in unreacted iso-C₄ paraffins;
recycling the stream rich in unreacted n-C₄ and n-C₅ paraffins from the disproportionation separation zone to the disproportionation reaction zone;
recycling the stream rich in unreacted iso-C₄ paraffins from the alkylation separation zone to the alkylation reaction zone; and
recovering at least one of the C₆₊ isoparaffin-rich stream the disproportionation separation zone and the alkylate-rich stream from the alkylation separation zone.

2. The process of claim 1 wherein the disproportionation mixture further comprises C₃₋ product, and wherein separating the disproportionation mixture into at least the iso-C₄-rich stream, the C₆₊ isoparaffin-rich stream, and the stream rich in unreacted n-C₄ and n-C₅ paraffins comprises separating the disproportionation mixture into at least the iso-C4-rich stream, the C₆₊ isoparaffin-rich stream, the stream rich in unreacted n-C₄ and n-C₅ paraffins, and a stream rich in C₃₋ product, and further comprising:
recovering the stream rich in C₃₋ product.

3. The process of claim 1 wherein the alkylation mixture further comprises C₃₋ product, and wherein separating the alkylation mixture into at least the alkylate-rich stream, and the stream rich in unreacted iso-C₄ paraffins comprises separating the alkylation mixture into at least the alkylate-rich stream, the stream rich in unreacted iso-C₄ paraffins, and a stream rich in C₃₋ product, and further comprising:
recovering the stream rich in C₃₋ product.

4. The process of claim 1 wherein the alkylation mixture further comprises C₅ and C₆ product, and wherein separating the alkylation mixture into at least the alkylate-rich stream, and the stream rich in unreacted iso-C₄ paraffins comprises separating the alkylation mixture into at least the alkylate-rich stream, the stream rich in unreacted iso-C₄ paraffins, and a stream rich in C₅ and C₆ product, and further comprising:
recovering the stream rich in C₅ and C₆ product.

5. The process of claim 1 further comprising introducing make-up iso-C₄ into the alkylation reaction zone.

6. The process of claim 1 wherein at least one of the disproportionation catalyst and the alkylation catalyst comprises a solid acid catalyst.

7. The process of claim 6 wherein the solid acid catalyst comprises a refractory inorganic oxide having a metal halide dispersed thereon, and optionally a Group VIII metal component dispersed thereon.

8. The process of claim 1 wherein at least one of the disproportionation catalyst and the alkylation catalyst comprises a liquid acid catalyst.

9. The process of claim 8 wherein the liquid acid catalyst comprises HF, H₂SO₄, fluorosulfonic acids, or an acidic ionic liquid.

10. The process of claim 1 wherein the disproportionation reaction conditions comprise at least one of: a temperature in a range of about 50° C. to about 300° C., a pressure in a range of about 0.1 MPa to about 13.8 MPa.

11. The process of claim 1 wherein the alkylation reaction conditions comprise at least one of: a temperature in a range of about −30° C. to about 120° C., a pressure in a range of about 0.1 MPa to about 13.8 MPa.

12. The process of claim 1 wherein separating the disproportionation mixture into at least the iso-C₄-rich stream, the C₆₊ isoparaffin-rich stream, and the stream rich in unreacted n-C₄ and n-C₅ paraffins comprises fractionating the disproportionation mixture into at least the iso-C₄-rich stream, the C₆₊ isoparaffin-rich stream, and the stream rich in unreacted n-C₄ and n-C₅ paraffins in a fractionation column; or wherein separating the alkylation mixture into at least the alkylate-rich stream, and the stream rich in unreacted iso-C₄ paraffins comprises fractionating the alkylation mixture into at least the alkylate-rich stream, and the stream rich in unreacted iso-C₄ paraffins in a fractionation column; or both.

13. An integrated process for normal paraffin alkylation comprising:
introducing a feed comprising n-C₄ and n-C₅ paraffins to a disproportionation reaction zone in the presence of a disproportionation catalyst under disproportionation reaction conditions to form a disproportionation mixture comprising iso-C₄ and C₆₊ disproportionation products and unreacted n-C₄ and n-C₅ paraffins;
separating the disproportionation mixture in a disproportionation separation zone into at least an iso-C₄-rich stream, a C₆₊ isoparaffin-rich stream, and a stream rich in unreacted n-C₄ and n-C₅ paraffins;
introducing the iso-C₄-rich stream and an olefin feed stream comprising at least one of ethylene, propylene and butenes into an alkylation reaction zone in the presence of an alkylation catalyst under alkylation reaction conditions to produce an alkylation mixture comprising alkylate, C₅ and C₆ product, and unreacted iso-C₄ paraffins;
separating the alkylation mixture in an alkylation separation zone into at least an alkylate-rich stream, a stream rich in C₅ and C₆ product, and a stream rich in unreacted iso-C₄ paraffins;
recycling the stream rich in unreacted n-C₄ and n-C₅ paraffins from the disproportionation separation zone to the disproportionation reaction zone;
recycling the stream rich in unreacted iso-C₄ paraffins from the alkylation separation zone to the alkylation reaction zone; and
recovering at least one of the C₆₊ isoparaffin-rich stream from the disproportionation separation zone, the alkylate-rich stream from the alkylation separation zone, and the stream rich in C₅ and C₆ product from the alkylation separation zone.

14. The process of claim 13 wherein the disproportionation mixture further comprises C₃₋ product, and wherein separating the disproportionation mixture into at least the iso-C₄-rich stream, the C₆₊ isoparaffin-rich stream, and the stream rich in unreacted n-C₄ and n-C₅ paraffins comprises separating the disproportionation mixture into at least the iso-C₄-rich stream, the C₆₊ isoparaffin-rich stream, the stream rich in unreacted n-C₄ and n-C₅ paraffins, and a stream rich in C₃₋ product, and further comprising:
recovering the stream rich in C₃₋ product;
or
wherein the alkylation mixture further comprises C₃₋ product, and wherein separating the alkylation mixture into at least the alkylate-rich stream, and the stream rich in unreacted iso-C₄ paraffins comprises separating the alkylation mixture into at least the alkylate-rich stream, the stream rich in unreacted iso-C₄ paraffins, and a stream rich in C₃₋ product, and further comprising:
recovering the stream rich in C₃₋ product;
or both.

15. The process of claim 13 further comprising introducing make-up iso-C₄ into the alkylation reaction zone.

16. The process of claim 13 wherein at least one of the disproportionation catalyst and the alkylation catalyst comprises a solid acid catalyst.

17. The process of claim 13 wherein at least one of the disproportionation catalyst and the alkylation catalyst comprises a liquid acid catalyst.

18. The process of claim 13 wherein the disproportionation reaction conditions comprise at least one of: a temperature in a range of about 50° C. to about 300° C., a pressure in a range of about 0.1 MPa to about 13.8 MPa; or wherein the alkylation reaction conditions comprise at least one of: a temperature in a range of about −30° C. to about 120° C., a pressure in a range of about 0.1 MPa to about 13.8 MPa.

19. The process of claim 13:
wherein separating the disproportionation mixture into at least the iso-C₄-rich stream, the C₆₊ isoparaffin-rich stream, and the stream rich in unreacted n-C₄ and n-C₅ paraffins comprises fractionating the disproportionation mixture into at least the iso-C₄-rich stream, the C₆₊ isoparaffin-rich stream, and the stream rich in unreacted n-C₄ and n-C₅ paraffins in a fractionation column; or
wherein separating the alkylation mixture into at least the alkylate-rich stream, the stream rich in C₅ and C₆ product, and the stream rich in unreacted iso-C₄ paraffins comprises fractionating the alkylation mixture into at least the alkylate-rich stream, the stream rich in C₅ and C₆ product, and the stream rich in unreacted iso-C₄ paraffins in a fractionation column; or
both.

20. An apparatus for normal paraffin alkylation comprising:
a disproportionation reaction zone having an inlet and an outlet;
a disproportionation separation zone having an inlet and at least one outlet, the inlet of the disproportionation separation zone being in fluid communication with the outlet of the disproportionation reaction zone, an outlet of the disproportionation separation zone being in fluid communication with the inlet of the disproportionation reaction zone;
an alkylation reaction zone having at least one inlet and at least one outlet, an inlet of the alkylation reaction zone being in fluid communication with an outlet of the disproportionation separation zone;
an alkylation separation zone having an inlet and at least one outlet, the inlet of the alkylation separation zone being in fluid communication with the outlet of the alkylation reaction zone, an outlet of the alkylation separation zone being in fluid communication with the inlet of the alkylation reaction zone.
